(19) United States
(12) Patent Application Publication
(10) Pub. No.: US 2025/0176983 A1
(43) Pub. Date: Jun. 5, 2025

(54) Magnetic Snare Endovascular Catheter System for Central Venous Stenosis

(71) Applicant: UNM Rainforest Innovations, Albuquerque, NM (US)

(72) Inventors: Jonathan Owen, Albuquerque, NM (US); Claire E. O'Malley, Albuquerque, NM (US); Addie Voorhees, Albuquerque, NM (US); Alex Bender, Albuquerque, NM (US); Adan Casados, Albuquerque, NM (US); Simeon Moya, Albuquerque, NM (US); Andrew Fisher, Albuquerque, NM (US)

(73) Assignee: UNM Rainforest Innovations, Albuquerque, NM (US)

(21) Appl. No.: 18/840,471

(22) PCT Filed: Feb. 21, 2023
(86) PCT No.: PCT/US2023/062960

§ 371 Date: Aug. 21, 2024

Related U.S. Application Data
(60) Provisional application No. 63/312,278, filed on Feb. 21, 2022.
(60) Provisional application No. 63/417,663, filed on Oct. 19, 2022.
(60) Provisional application No. 63/432,679, filed on Dec. 14, 2022.

(51) Int. Cl. A61B 17/22; A61B 17/00

(52) U.S. Cl. CPC A61B 17/22; A61B 2017/00778; A61B 2017/00876; A61B 2017/22075; A61B 2017/22094

(57) ABSTRACT

A method, device and system having dual catheters each containing a large circular, circumferential and oppositely polar magnet at each catheter tip.

### RELATED APPLICATIONS

This application is a U.S. 371 National Phase of PCT/US2023/062960 filed on Feb. 21, 2023, which claims priority to U.S. Provisional Application Nos. 63/312,278 filed Feb. 21, 2022, 63/417,663 filed on Oct. 19, 2022 and 63/432,679, filed on Dec. 14, 2022, all of which are incorporated herein in their entirety.

### BACKGROUND OF THE INVENTION

Central venous occlusion is a common and challenging clinical problem encountered in patients who have had longstanding indwelling central venous catheters (CVC), such as those undergoing hemodialysis via a CVC or in patients with an indwelling CVC for chemotherapy. Symptoms from this condition include uncomfortable facial and arm swelling and the debilitating and rarely fatal superior vena cava syndrome. Central venous occlusion accounts for a major cause of vascular access failure in hemodialysis patients and can lead to termination of dialysis and death for dialysis patients if renal transplantation or peritoneal dialysis is not possible, as is often encountered.

Current techniques for recanalization of a central venous occlusion start first with attempts of unidirectional crossing of the occlusion with either a stiff or flexible 0.018 mm or 0.035 mm guidewire, directed from a venous sheath placed within a large vein such as the internal jugular vein or an arteriovenous fistula or graft. If the occlusion can be traversed with a guidewire, the occlusion can be dilated with balloon angioplasty technique and/or central venous stent deployment.

Unfortunately, in many cases, the central venous occlusion is not able to be traversed in a unidirectional fashion to allow for recanalization. In this situation, sharp needle or radiofrequency recanalization can be attempted with a technique where the central venous occlusion is approached bi-directionally, i.e. “above and below”, usually with one guidewire directed from a venous sheath placed within a large upper body vein such as the internal jugular vein or an arteriovenous fistula (AVF) or graft (AVG), and a second guidewire directed to the opposite side of the central venous occlusion, usually from a venous sheath placed within a femoral vein. Once the guidewires come within close approximation of one another, a guiding catheter can be advanced over the wire to the lesion to serve as a target for sharp recanalization. Via the opposite guidewire, a guiding catheter can be advanced to the occlusion, and the blunt end of a stiff guidewire can be used to puncture the occlusion. Alternately, if this is unsuccessful, a long venous angiosheath can be advanced to the lesion and using a transjugular liver access set designed for the Transjugular Intrahepatic Portosystemic Shunt procedure (TIPS). In this procedure, a sharp needle is used to puncture the central occlusion. A third described technique allows for a radiofrequency catheter to be advanced to the central lesion, where radiofrequency energy is then applied to “burn a hole” through the fibrotic lesion. Once a passageway has been created across the occlusion, the leading guidewire can be advanced across the lesion and a snare device can be inserted to “grab” the opposite guidewire a pull it across the lesion. After this is accomplished, the lesion can be addressed as would be in the unidirectional approach with dilatation using balloon angioplasty technique and/or central venous stent deployment.

The major limitation in the bi-directional approaches described above is that it is impossible to guarantee that the recanalization attempts will result in direct communication from one side of the vascular stenosis to the other while remaining within the venous system. Occasionally, these techniques result in puncture across the vein and into the extravascular space. This can result in massive hemorrhage, hemopericardium with cardiac tamponade, hemothorax, or retroperitoneal bleeding. These complications, if unable to be controlled with endovascular salvage techniques or immediate surgery, generally result in death. Because of these potential catastrophic consequences, many Endovascular Interventionalists are unwilling to attempt these techniques and these lesions often go untreated. New techniques to ensure accurate recanalization while minimizing risk of major hemorrhages are therefore needed.

Central Venous Occlusion (CVO) may also be a significant source of discomfort for many patients. CVO is damage to the veins due to “mechanical damage to the vessel walls from prior catheterization”. Essentially it is scarring caused by damage to the veins from long-term trauma. This can be very painful to a patient causing facial and upper arm swelling and in severe cases can compromise the airways or cause a patient to lose main access sites for dialysis. The current procedure to fix CVO involves a catheter directed balloon to form a bridge across the occlusion (or scar). Needles are used to break apart the occlusion and connect catheters from veins on either side of the occlusion. This can be a very risky procedure as the surgeon is viewing the needles on the two-dimensional platform and the human body is in three-dimensional space.

### BRIEF SUMMARY OF THE INVENTION

In one embodiment, the present invention provides a method, device and system having dual catheters each containing a large circular, circumferential and oppositely polar magnet at each catheter tip. The circular magnet has a central lumen, which creates a magnetic attachment, alignment or pairing to the opposite catheter with equal approximation or alignment of opposing lumens. This allows for direct guided passage of guidewires and other intravascular instruments from one body compartment or vessel directly into the other through the lumens. This “magnetic snare” allows for direct approximation between compartments and eliminates the potential for miscalculation and puncture of adjacent structures.

In another aspect, the present invention consists of inserting a magnetic tip into an already inserted catheter. This magnetic tip would attract a Chiba Needle, already used in the procedure, through the occlusion resulting in an increase in safety of the procedure as the magnetic insert and Chiba needle would self-align when in close contact. The embodiments of the present invention may be used for CVO patients as well as other procedures needing the connection of two catheters.

In another aspect, the present invention consists of device and method involving the insertion of a device with a magnetic tip into an already inserted catheter. This magnetic tip would be used to attract and align either a second magnetic device or an existing ferromagnetic tool through the occlusion.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1 shows a first embodiment of the present invention.

FIG. 2 shows a second embodiment of the present invention.

FIG. 3 shows an instrument deploying one or more rigid or flexible magnets.

FIG. 4 shows how the instrument shown in FIG. 3 may be magnetically paired with another instrument.

FIG. 5 shows another embodiment using a steel tube to focus the magnetism of an enclosed magnet.

FIG. 6 shows an embodiment of the present invention concerning a magnetic snare endovascular instrument for use in treating central venous occlusions.

FIG. 7 shows how the embodiment shown in FIG. 6 may be curved and bended.

FIG. 8 shows how a spacer may be used with the embodiments of the present invention.

FIG. 9 shows an exploded view of a spacer that may be used with the embodiments of the present invention.

FIG. 10 shows an embodiment of the present invention deploying one or more electromagnets.

FIG. 11 shows an embodiment of the present invention deploying an electromagnet introducer.

FIG. 12 shows an embodiment of the present invention deploying a heater.

FIG. 13 shows an embodiment of the present invention deploying an extendable and retractable heater.

FIG. 14 illustrates a system including pair of instruments configured to pair magnetically to clear a vein or other occlusions.

FIG. 15 illustrates the system shown in FIG. 14 in a paired position.

FIG. 16A illustrates an embodiment of the present invention providing a cutting tool.

FIG. 16B illustrates the instrument shown in FIG. 16A along with a corresponding instrument prior to magnetically pairing.

FIG. 17 illustrates an embodiment of the present invention providing mating and interlocking distal ends.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

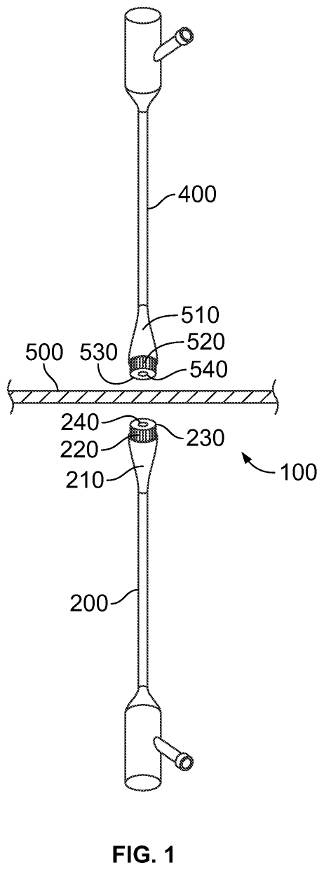

In one embodiment, as shown in FIG. 1, the present invention provides a method, device, and system 100 having dual catheters 200, 400 each containing at distal ends 210, 510, opposing polar magnets 220, 520. In a preferred embodiment, the distal ends are circular and have matching edges 230, 530. Each catheter has a central lumen 240, 540 located inside the catheter.

Use of magnets 220, 520 allow lumens 240, 540 to be magnetically attached, aligned or paired or at least be in close proximity of each other. This substantially aligns the openings of the opposingly located lumens. In a preferred embodiment, edges 230, 530 match-up as do lumens 240,540.

The approximate matching or alignment of lumens 240,540 allows for direct guided passage of guidewires, other intravascular instruments, or devices from lumen 240 of catheter 200 through a body compartment or vessel directly into lumen 540 of catheter 500, or vice-versa.

Using magnetic attraction or a “magnetic snare” allows the two catheters to be joined, aligned or paired together without physically touching through body tissue or other substance. This, in-turn, sufficiently aligns the lumens to create a common passageway that allows a device to pass through a first, transmitting lumen in a guided manner so that the device is directed into a second, receiving lumen. The direct or near approximation between lumens eliminates the potential for miscalculation and puncture of adjacent structures.

In other embodiments, to increase the possibility of a successful transfer of a device between lumens, the ends of the lumens may be slightly flared, conical in configuration, or generally opened. Other ways to increase the pairing of the ends include the use of a prong and socket, external and internal threads, as well as other means to releasably join to ends together. In other embodiments, as shown in FIG. 17, each catheter end has a mating shape such as body 2000 with tapered end 2005, which may be frustoconical in shape, that is configured to mate with and nest within opening 2011 that is defined by flared wall 2012. Flared wall 2012 is configured to match the shape of the receiving end thereby enabling the two ends to mate. Mating of the ends results in tapered end 2005 being securely nested within opening 2011. In this configuration the smaller end of taper 2005 is guided into the larger initial opening until it is centered inside of opening 2011 where it is locked in place preventing any side-to-side movement that may result in the misalignment of the connecting lumens or connecting instruments.

In one method of using the present invention concerning a central venous occlusion, the bidirectional approach of the above described embodiments of the present invention permit catheter placement above the lesion via a sheath in a large vessel such as the internal jugular vein or hemodialysis AVF or AVG, and below the lesion via a sheath in the femoral vein would still be used; however, once guidewires were advanced to close approximation of one another on either side of the stenotic lesion, the dual magnetic catheters may be advanced over the guidewires to either side of the central occlusion and attached or paired to one another. The magnetic attraction sufficiently aligns the central lumens to allow a wire to be advanced from a lumen of one catheter directly to the lumen of another catheter. This reduces the risk of extravascular puncture and hemorrhage. After successful puncture of the lesion, blockage or matter, the opposite guiding instrument may be magnetically dragged across the lesion to then allow for balloon angioplasty of the stenosis and endovascular stenting.

Similarly, if the opening created across the vascular stenosis was too small to magnetically drag the opposite instrument across the stenosis, a steel guidewire could be advanced via one catheter and via magnetic attraction then pulled across the stenotic lesion. Alternatively, radiofrequency energy could also be applied at one instrument tip as well to create the puncture across the stenotic lesion, and again one instrument could then be magnetically dragged to the other side following recanalization.

Although the above-described embodiments of the present invention have been primarily described in connection with use concerning a central venous occlusion, the present invention may be used for other clinical indications, including but not limited to other vascular compartment lesions such as congenital heart defects (atrial septal defects, ventricular septal defects, patent foramen ovale), as well as crossing luminal structures of solid organs, as in the case of percutaneous gastrostomy tube placement.

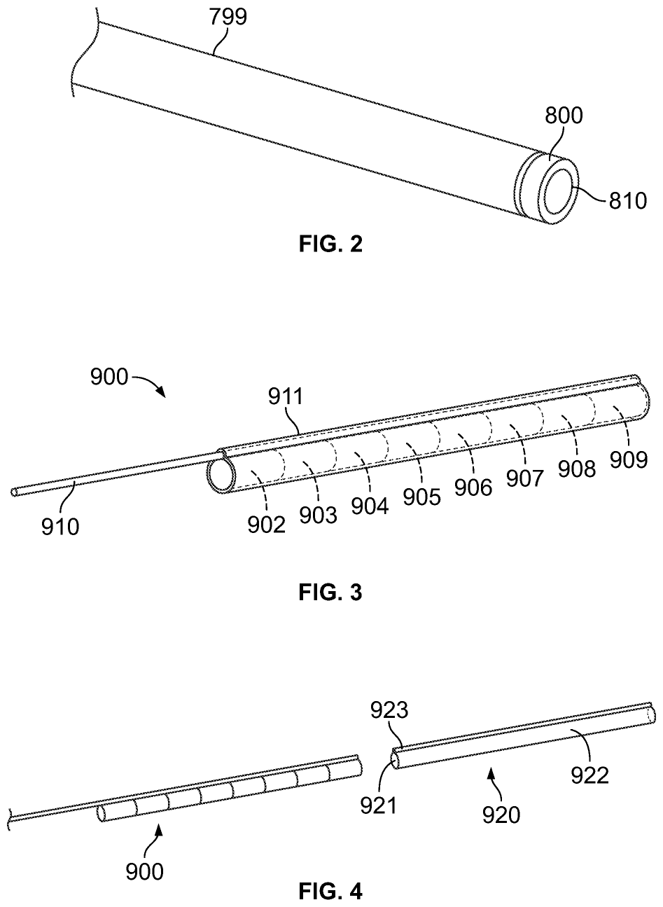

In another aspect, as shown in FIG. 2, the present invention concerns a catheter 799 with magnet 800 installed at or near tip 810. The magnetic tip design, as a result of using one or more magnets 800 of opposite polarities on the interfacing ends of the catheters used to clear occlusions. The goal is to use the magnetic attraction between catheter tips to self-align themselves when approaching a blockage with ease.

In another aspect, the present invention concerns an instrument 900 deploying one or more rigid or flexible magnets or segments 902-909 inside a flexible sheath as shown below in FIG. 3. This embodiment extends the reach of the magnetic field in the axial direction by lengthening the magnet. Also included is guidewire 910 which is attached to the outer surface of instrument 900 to form a spline 911.

As shown in FIG. 4, instrument 900 is configured to magnetically pair with instrument 920. instrument 920 is comprised of an elongated solid magnet 921 instead of a plurality of connected individual magnet segments. Elongating the magnet in this manner increases the axial magnetic force.

In yet another embodiment, as shown in FIG. 4, the magnetic field may be concentrated by enclosing one or more magnets such as magnet 921 in steel tube 922. In a preferred embodiment, tube 922 surrounds magnet 921 on its sides and one end leaving distal end 923 of magnet 921 uncovered by steel. This embodiment works by shorting the magnetic field causing it to focus its magnetism in one direction more strongly towards distal end 923.

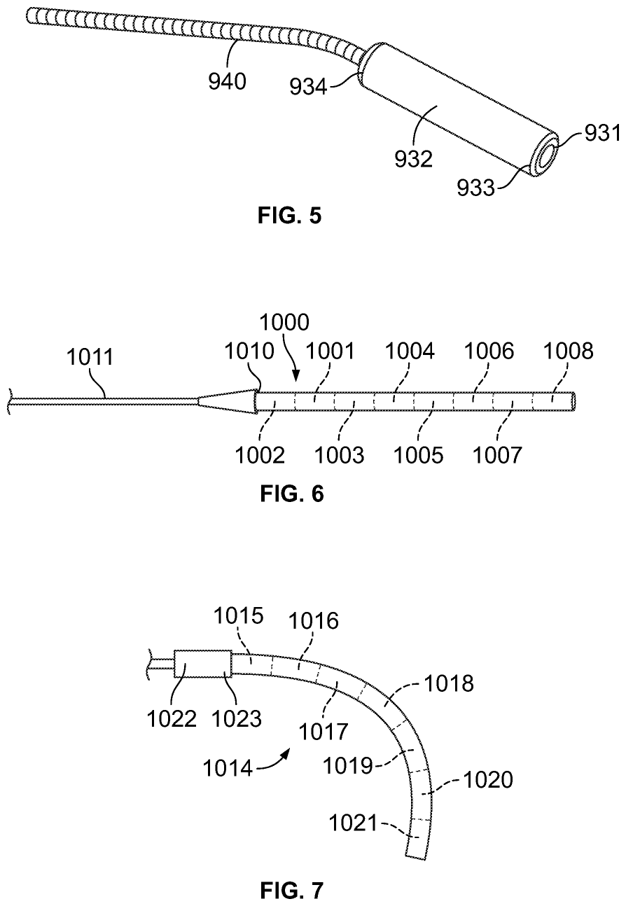

FIG. 5 shows another embodiment wherein tube 932 surrounds magnet 931 on its sides and one end 934 leaving distal end 933 of magnet 931 uncovered by steel. This embodiment utilizes a single magnet 931 that is 0.375 inches thick and 0.1 inches in diameter. The magnetic field is concentrated by steel tube 932 surrounding magnet 932 on its sides and one end 934. This embodiment also works by shorting the magnetic field causing it to focus its magnetism in one direction more strongly than the others at distal end 933. This embodiment is constructed of hypodermic tubing with a magnet stuck inside and the tubing crimped in place over the magnet. Steel wire 940 is bent at a right angle placed inside the tube with epoxy and crimped in place as well.

In another embodiment, as shown in FIG. 6, the present invention concerns a magnetic snare endovascular instrument 1000 for central venous occlusions having a plurality of magnets 1002-1008 located in a flexible tube 1010 attached to a wire 1012. In other aspects, the design may be comprised of a plurality of 0.25-inch magnets.

Enclosing a plurality of magnets in a flexible tube creates an ability for the instrument to make turns necessary for a number of applications including intravenous work. FIG. 7 shows how instrument 1014 is comprised on an elongated magnet may be made of smaller magnets 1015-1021 that are placed end to end. This was accomplished by placing many magnets aligned end to end alongside a guidewire 1022 with a small amount of silicone adhesive and all placed inside of heat shrink tubing 1023. This embodiment of the present invention has the ability to make a 90° turn over its length as shown in FIG. 7.

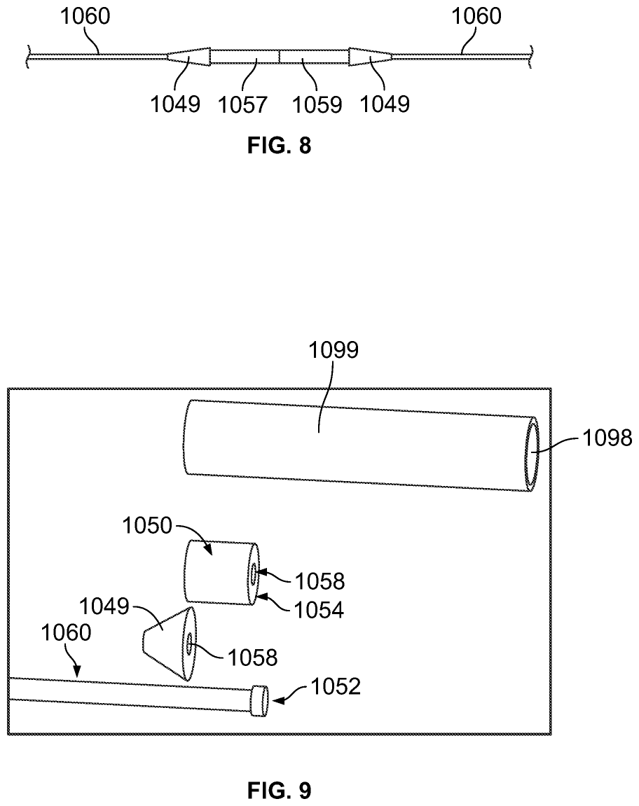

To promote the connection between a guidewire and the instrument, as shown in FIGS. 8 and 9, a spacer comprised of endcap 1049 and barrel 1050 which are affixed together may be provided. End cap 1049 and barrel 1050 have an opening 1058 therein which is sized to receive guidewire 1060. Guidewire 1060 includes boss 1052 that engages surface 1054 of barrel 1050 which prevents the guidewire from being pulled through the opening. Guidewire 1060 is inserted into end cap 1049 and barrel 1050 spacer and locked in place such as being peened in place. The guidewire could also be inserted in a way that allows for the bending of the guidewire such that it cannot back out through the hole such as bending at a 90° angle instead of or in addition to peening. Barrel 1050 is inserted into opening 1098 of instrument 1099 and affixed therein.

FIG. 8 shows how the instruments described above may be magnetically paired. As an exemplar, FIG. 8 shows instruments 1057 and 1059 are pair and aligned by magnetic attraction.

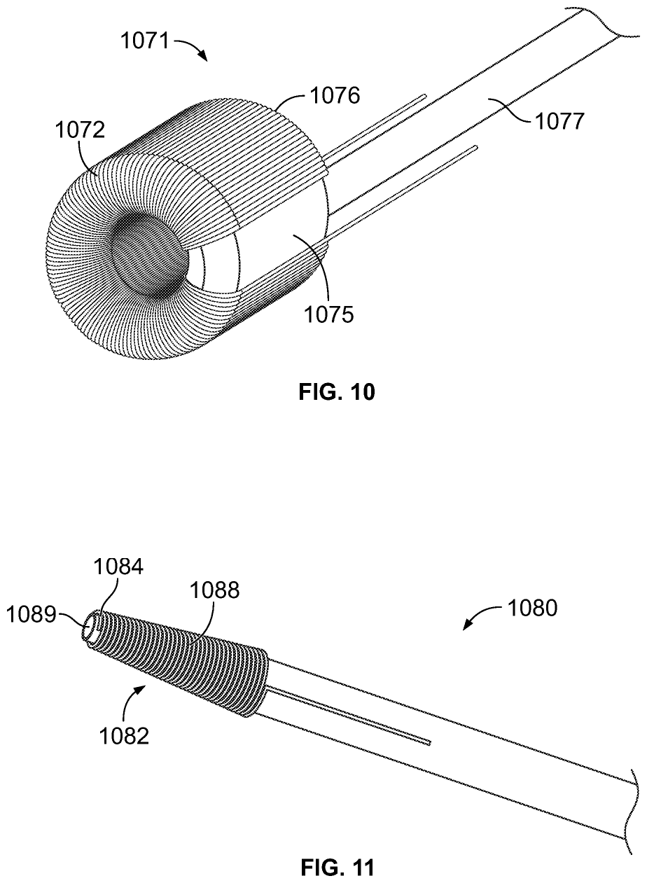

In another aspect, the present invention concerns an instrument 1071 deploying one or more electromagnets 1072 as shown in FIG. 10. This embodiment uses a toroidal electromagnet due to the fact that they have the strongest magnetic flux for their size. This embodiment uses steel core 1075 wrapped with an enameled copper wire 1076 with a hole drilled in the bottom allowing for the insertion of stainless-steel wire 1077. The entire tip is coated in a thin layer of epoxy resin to preserve the integrity of the magnet during use, and lead wires connect to a controller outside the catheter.

In another aspect, the present invention concerns an instrument 1080 deploying an electromagnet introducer 1082 as shown in FIG. 11. This embodiment incorporates an electromagnet, such as a solenoid electromagnet 1084 at the tip of the introducer that goes through the central venous catheter. The tip of the introducer may be made from some sort of ferrous metal to strengthen the force of the magnet and to concentrate the flux in a desired direction. Coils 1088 wrapped around the tip are made of copper wire. The electromagnet may be connected to a controller outside the catheter. There is a passageway 1089 for a guidewire to go through as in a standard introducer.

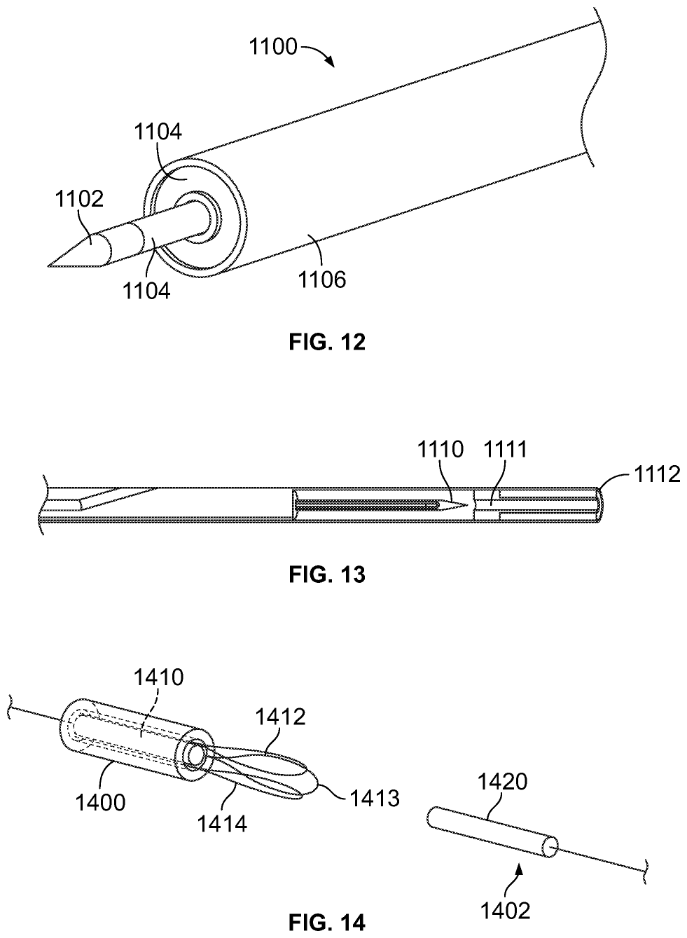

In another aspect, the present invention concerns an instrument 1100 having heater 1102 as shown in FIG. 12. This embodiment uses a heated device 1102 at the end of a guidewire 1104 that can be fed through the guidewire lumen 1104 of tubing 1106 of the instrument and manipulated by the user.

In another aspect, the present invention concerns a heater 1110 as shown in FIG. 13. This embodiment maintains the guidewire passage 1111 while incorporating the heated device into the distal tip of the instrument. This requires the heated device 1110 to have the capability of being extended and retracted out of the end of the distal tip 1112 through lumen 1111. In both designs shown in FIGS. 11 and 12, the heated device is heated using resistive heating, although other options of heating, such as radiofrequency energy, remain as potential options for heating and ablation of the occlusion.

Magnet with Snare

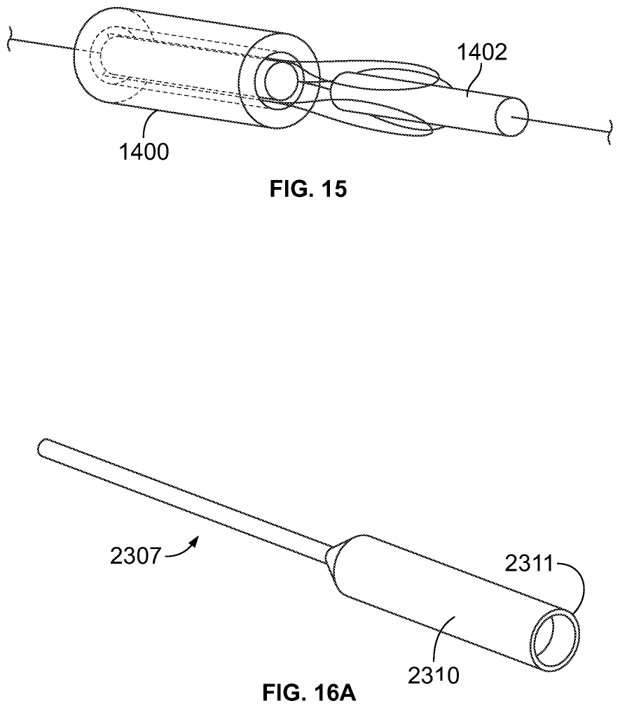

In another embodiment, the present invention concerns a magnet with snare design that utilizes the concept of the snare shaped catheter insert to help clear blockages in the veins as well as the magnetic connection to enable both sides of the instrument to connect successfully. As shown in FIGS. 14 and 15, this system includes a first instrument 1400 and 1402 which are adapted to pair magnetically. Instrument 1400 includes a magnet 1410 surrounded by flexible loop snares 1412-1414, which may be made of wire, all attached and protected with tubing such as heat shrink or metal tubing. Instrument 1402 includes magnet 1420.

The snare side of the device (instrument 1400) may feed down one end of the catheter toward the blocked vein, while the plain magnet (instrument 1402 without snares) would be fed up the catheter on the other end of the blockage. The magnetic force would pull the two components toward each other, while the snare end clears the entire vein while the magnets move towards each other and eventually connect with the distal end of instrument 1402 being located an opening defined by the loops as shown in FIG. 15. This would result in a clear vein with minimal damage.

### Single Magnet Cutter

This embodiment can be incorporated with the single magnet rigid design that has been previously described above. This design utilizes the entirety of the single magnet rigid design while extending the shielding around the magnet forward allowing the hypodermic tubing to be able to cut with guidance from a second instrument magnet on the other side of the material.

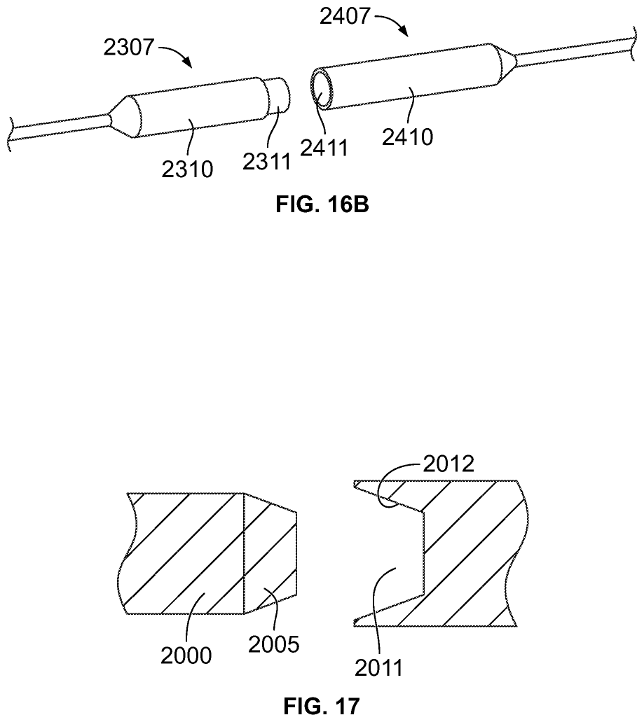

In one preferred embodiment of the magnetic cutter, the present invention provides a first instrument 2307 having a magnetic body 2310 having a distal cutting edge 2311 as shown in FIGS. 16A and 16B. Also provided a second instrument 2407 having a magnetic body 2410 defining chamber 2411. having a distal cutting edge 2311 as shown in FIGS. 16A and 16B. In use, this embodiment may be used to cut and remove materials such as occlusions by manually and magnetically drawing cutting edge 2311 through the material towards instrument 2407 and into chamber 2411. Once instruments 2307 and 2407 are paired, chamber 2411 is capped by instrument 2307 and the material inside is collected and contained. The collected materials are prevented from being further disbursed and may be safely removed or retained for other uses such as for a biopsy.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above-described embodiments, methods, and examples, but by all embodiments and methods within the scope and spirit of the disclosure.

What is claimed is:

1. A catheter system comprising: first and second catheters, each catheter having a distal end including a magnet, said magnet of said first catheter is the polar opposite of said magnet used with said second catheter, each catheter having a lumen, and said lumens are substantially aligned when said catheters are magnetically paired.

2. The system of claim 1 wherein said distal ends are circular.

3. The system of claim 1 wherein said distal ends are mirror-images.

4. The system of claim 1 wherein said lumens are centrally located.

5. The system of claim 4 wherein one of said distal ends is tapered and another of said distal ends has an opening configured to receive said tapered end and to lock said tapered end in place when said catheters are joined together.

6. The system of claim 5 wherein said tapered end is frustoconical in shape and said opening is configured to match said frustoconical shape.

7. The system of claim 1 wherein at least one of said magnets are enclosed in a steel tubing.

8. The system of claim 1 wherein said steel tubing does not cover the distal end of said magnet.

9. The system of claim 1 wherein at least one of said magnets is bendable.

10. The system of claim 1 wherein said bendable magnet is comprised of a plurality of segments.

11. A medical system comprising: first and second instruments, each instrument having a distal end including a magnet, said magnet of said first instrument is the polar opposite of said magnet used with said second instrument, and magnets are substantially aligned when said instrument are magnetically paired.

12. The system of claim 11 wherein said distal ends are circular.

13. The system of claim 11 wherein said distal ends are mirror-images.

14. The system of claim 11 wherein said lumens are centrally located.

15. The system of claim 11 wherein one of said distal ends is tapered and another of said distal ends has an opening configured to receive said tapered end and to lock said tapered end in place when said catheters are joined together.

16. The system of claim 15 wherein said tapered end is frustoconical in shape and said opening is configured to match said frustoconical shape.

17. The system of claim 11 wherein one of said magnets is an electromagnet.

18. The system of claim 11 wherein one of said ends includes a heater.

19. The system of claim 18 wherein said heater is extendable and retractable.

20. The system of claim 11 wherein said distal end of said first instrument includes one or more loops defining an opening and said distal end.

21. The system of claim 20 wherein said loops enclose said magnet of said second instrument when said instruments are paired.

22. The system of claim 11 wherein at least one of said magnets are enclosed in a steel tubing.

23. The system of claim 11 wherein said steel tubing does not cover the distal end of said magnet.

24. The system of claim 11 wherein at least one of said magnets is bendable.

25. The system of claim 11 wherein said bendable magnet is comprised of a plurality of segments.

26. The system of claim 11 wherein one of said distal ends has a cutting end and another of said distal ends has an opening configured to receive said cutting end.

27. The system of claim 26 wherein receiving end defines a chamber and said chamber is capped by said cutting end when said instruments are magnetically paired.

28. A method of advancing a device from a first lumen to a spaced apart second lumen comprising the steps of: providing first and second catheters, each catheter having a distal end including a magnet, the magnet of the first catheter is the polar opposite of the magnet used with the second catheter, each catheter having a lumen; and advancing said distal end of each catheter close to one another until said magnets pair said ends together to aligned said lumens so as to permit the passage of the device from said first lumen to said second lumen.

29. The method of claim 28 wherein the method is used in connection with a central venous occlusion.

30. The method of claim 28 wherein the bidirectional approach of the distal ends permits catheter placement above the lesion via a sheath in a large vessel such as the internal jugular vein or hemodialysis AVF or AVG, and below the lesion via a sheath in the femoral vein.

31. The method of claim 28 wherein once guidewires are advanced to close approximation of one another on either side of the stenotic lesion, the dual magnetic catheters may be advanced over the guidewires to either side of the central occlusion and attached or paired to one another wherein the magnetic attraction sufficiently aligns the central lumens to allow an instrument to be advanced from a lumen of one catheter directly to the lumen of another catheter even though there may be a spaced distance between the two ends.

32. The method of claim 28 wherein after successful puncture of the lesion, blockage or matter, the opposite guiding instrument is magnetically dragged across the lesion to allow for balloon angioplasty of the stenosis and endovascular stenting.

33. The method of claim 28 further wherein, if the opening created across the vascular stenosis is too small to magnetically drag the opposite instrument across the stenosis, a steel guidewire is advanced via one catheter and via magnetic attraction then pulled across the stenotic lesion.

34. The method of claim 28 further wherein, if the opening created across the vascular stenosis is too small to magnetically drag the opposite instrument across the stenosis, radiofrequency energy is applied at one instrument tip as well to create the puncture across the stenotic lesion, and again one instrument is then magnetically dragged to the other side following recanalization.
